(19) United States
(12) Patent Application Publication
(10) Pub. No.: US 2011/0034771 A1
(43) Pub. Date: Feb. 10, 2011

(54) Endoscope resilient deflection section frame

(75) Inventor: Gregory S. Konstorum, Stamford, CT (US)

Correspondence Address: HARRINGTON & SMITH, 4 RESEARCH DRIVE, Suite 202, SHELTON, CT, 06484-6212, US

(73) Assignee: Gyrus ACMI, Inc.

(21) Appl. No.: 12/462,722

(22) Filed: Aug. 7, 2009

(51) Int. Cl. A61B 1/008; B23P 11/00

(52) U.S. Cl. 600/141; 29/428

(57) ABSTRACT

An endoscope deflection section frame including a plurality of rings and leaf springs connecting the rings to one another. Each ring includes front and rear ends with slots, wherein the slots extend into front and rear sides of the rings entirely through a wall of the ring between an interior of the wall at a central channel of the ring and an opposite exterior of the wall. The leaf springs each have opposite ends located in the slots of respective adjacent ones of the rings.

### BACKGROUND OF THE INVENTION

1. Field of the Invention

The invention relates to an endoscope and, more particularly, to a deflection frame section of an endoscope.

2. Brief Description of Prior Developments

U.S. Pat. No. 5,873,817 discloses an endoscope with a resilient deflectable section including rings connected by flexible connection members located in holes of the rings. There are also other known articulation system constructions such as disclosed in U.S. Patent Publication No. 2005/0245789 A1 including riveted systems where deflection rings are connected by rivets and a design where deflection rings are connected together by balls having holes orientated parallel to the longitudinal axis of the deflection system; all rings and balls being held together with resilient members (wire or cable) passing through corresponding holes in the rings spherical seats.

There is a desire to provide a shaft frame for an endoscope which has better torque resistance compared to slotted tube designs, such as described in U.S. Pat. Nos. 6,749,560 and 6,780,151, but with less complicated configurations compared to the riveted and ball constructions noted above. However, there is still the desire to provide resilient properties and internal low profile space of the slotted tube designs.

### SUMMARY

The following summary is merely intended to be exemplary. The summary is not intended to limit the scope of the claimed invention.

In accordance with one aspect of the invention, an endoscope deflection frame member is provided comprising a one-piece ring having front and rear sides adapted to be connected to adjacent rings to form an endoscope shaft frame. At least one of the sides comprises slots extending into the at least one side. The slots extend entirely through a wall of the ring between an interior of the wall at a central interior channel of the ring and an opposite exterior of the wall. The slots are sized and shaped to matingly receive opposite ends of a leaf spring therein to attach the ring to one of the adjacent rings.

In accordance with another aspect of the invention, an endoscope deflection frame is provided including a plurality of rings and leaf springs connecting the rings to one another. Each ring includes front and rear ends with slots, wherein the slots extend into front and rear sides of the rings entirely through a wall of the ring between an interior of the wall at a central channel of the ring and an opposite exterior of the wall. The leaf springs each have opposite ends located in the slots of respective adjacent ones of the rings.

In accordance with another aspect of the invention, a method is provided comprising positioning a leaf spring between two rings, wherein the rings comprise opposing faces each having a slot, wherein the slots extend entirely through a wall of each respective ring between an interior of the wall at a central channel of the respective ring and an opposite exterior of the wall, wherein the leaf spring comprises opposite ends positioned in the respective slots; and fixedly attaching the leaf spring to the rings at the slots.

### BRIEF DESCRIPTION OF THE DRAWINGS

The foregoing aspects and other features of the invention are explained in the following description, taken in connection with the accompanying drawings, wherein:

FIG. 1 is a side elevational view of an endoscope incorporating features of the present invention;

FIG. 2 is perspective view of a portion of a frame of the deflection section of the endoscope shown in FIG. 1;

FIG. 3 is an elevational side view of the frame shown in FIG. 2;

FIG. 4 is an enlarged view of area A sown in FIG. 3;

FIG. 5 is partial enlarged perspective view of a portion of the frame shown in FIGS. 2-4;

FIG. 6 is a perspective view of one of the frame member rings and leaf springs at one end;

FIG. 7 is a partial cross sectional view of the leaf springs and ring shown in FIG. 6;

FIG. 8 is a perspective view of one of the leaf springs shown in FIG. 6;

FIG. 9 is an elevational side view of the leaf spring shown in FIG. 8;

FIG. 10 is a cross sectional view of the leaf spring shown in FIG. 9;

FIG. 11 is a perspective view of a mandrel used in manufacture of the frame;

FIG. 12 is an enlarged view of the end of the mandrel shown in FIG. 11 with a partial cut away section;

FIG. 13 is a perspective view of a portion of an alternate embodiment of the frame shown in FIG. 5; and

FIG. 14 is a cross sectional view of an alternate embodiment of the leaf spring shown 10.

### DETAILED DESCRIPTION OF EMBODIMENTS

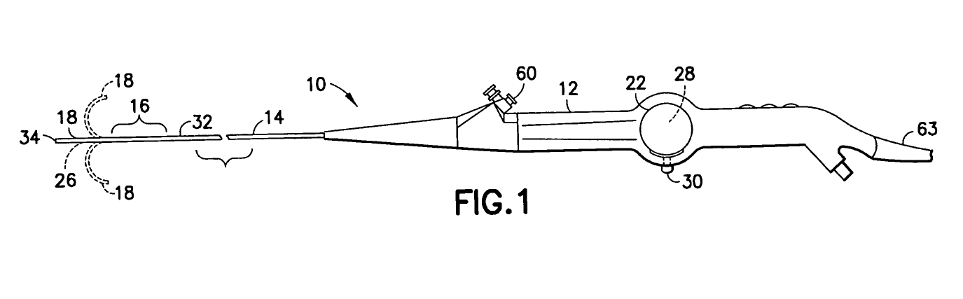

Referring to FIG. 1, there is shown a side view of an endoscope 10 incorporating features of the invention. Although the invention will be described with reference to the example embodiments shown in the drawings, it should be understood that the invention can be embodied in many alternate forms of embodiments. In addition, any suitable size, shape or type of elements or materials could be used.

The endoscope 10 is a ureteroscope. However, in alternate embodiments the endoscope could be any suitable type of endoscope. The endoscope 10 generally comprises a handle or control 12 and a flexible or semi-flexible shaft 14 connected to the handle 12. A deflection section is located at a distal end of the shaft 14 which, in this example embodiment, includes a passive deflection section 16 and an active deflection section 18. A control system 22 to control the active deflection section 18 extends from the handle 12 to the active deflection section 18. The control system 22 can comprise, for example, a pair of control wires, two wire sheaths, and an actuator 28. One end of the wires are connected to the actuator 28 and a second end of the wires are connected to the distal end of the active deflection section 18.

In the preferred embodiment, the handle 12 has a user operated slide or lever 30. The lever 30 is connected to the actuator 28. The actuator 28 is adapted to pull and release the two wires of the control system 22. When the lever 30 is moved by the user, the actuator 28 is moved. The actuator 28 may be a drum or pulley, for example, rotatably connected to the handle 12 to pull one wire while releasing the other. In an alternate embodiment, the actuator may be any suitable type of device, such as a rocker arm adapted to pull and release the wires of the control system 22. In another alternate embodiment, where the control system may have two or more pairs of control wires, the handle can have additional actuators and corresponding controls to drive the additional pairs of control wires to bend the deflection section in different plane(s). In still other alternate embodiments, the handle may have knobs with rack and pinion mechanisms or other suitable user operated controls for the control system.

The shaft 14 is cantilevered from the handle 12. The flexible shaft 14 includes the control wires of the control system 22, a fiber optical image bundle or a video sensor electrical cable, a working channel, and a fiber optical illumination bundle or electrical wires to illumination LEDs or lights at the objective head 34. A port 60 for inserting accessory instruments (not shown) into the working channel is located on the handle 12. The handle 12 also has an electrical cable 63 for connection to another device, such as a video monitor. In an alternate embodiment, instead of the cable 63, the endoscope could have an eyepiece. In alternate embodiments, the flexible shaft may house different systems within.

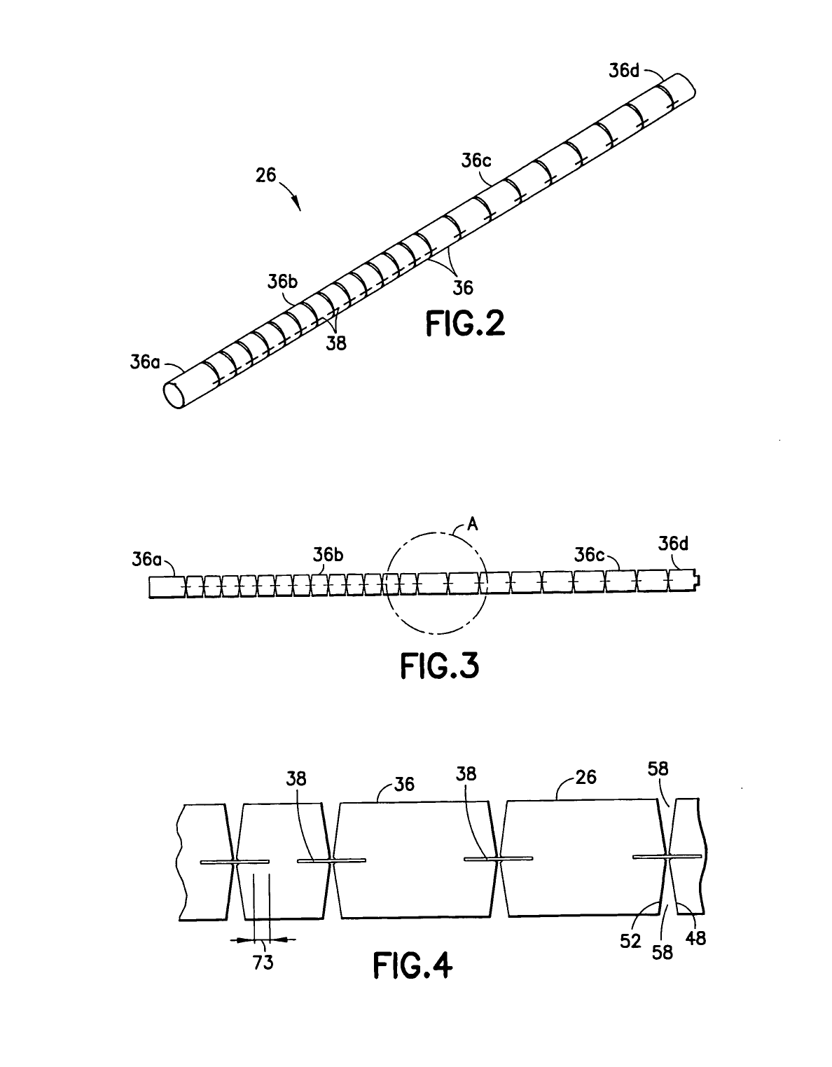

The deflection section (or steering section) at the distal end of the shaft 14 generally comprises a frame 26, a cover 32 and the objective head 34. The cover 32 extends over both the shaft 14 and the deflection section. In an alternate embodiment, the deflection section could have a different softer cover. Referring also to FIGS. 2-6, at least one portion of the frame 26 generally comprises a plurality of rings 36 and connectors 38. The connectors 38 connect the rings 36 to adjacent rings. The rings 36 and connectors 38 are preferably comprised of a shape memory alloy material, such as Tinel or Nitinol. The rings and connectors are preferably comprised of the same material for good welding purposes. However, the rings and/or connectors could be comprised of another material such as stainless steel or plastic for example.

A shape memory alloy material can be used for its superelastic properties exhibited by the material's ability to deflect and resiliently return to its natural or predetermined position even when material strains approach 4%, or an order of magnitude greater than the typical yield strain of 0.4% giving rise to plastic deformation in common metals. Thus, the term “superelastic alloy” is used to denote this type of material. The wire sheaths may also be comprised of this type of material such as disclosed in U.S. Pat. No. 5,938,588 which is hereby incorporated by reference in its entirety.

The rings 36 each comprise a one-piece member, but could be comprised of multiple members. The portion of the frame 26 shown in FIGS. 2-3 is connected to the very distal end of the frame (not shown) of the shaft 14. However, features of the invention could be used at other locations of the shaft 14 or passive deflection section 16 as well. In order to provide different deflection radii, in this embodiment the portion comprises two different types of rings; short rings 36b and long rings 36c. The frame 26 also comprises an objective head coupler 36a for coupling the frame to the objective head, and a shaft coupler 36d for coupling the frame 26 to the frame of the shaft or the frame of the passive deflection section. However, in alternate embodiment more or less than two types of deflection rings could be provided. In addition, the rings could have different shapes and sizes.

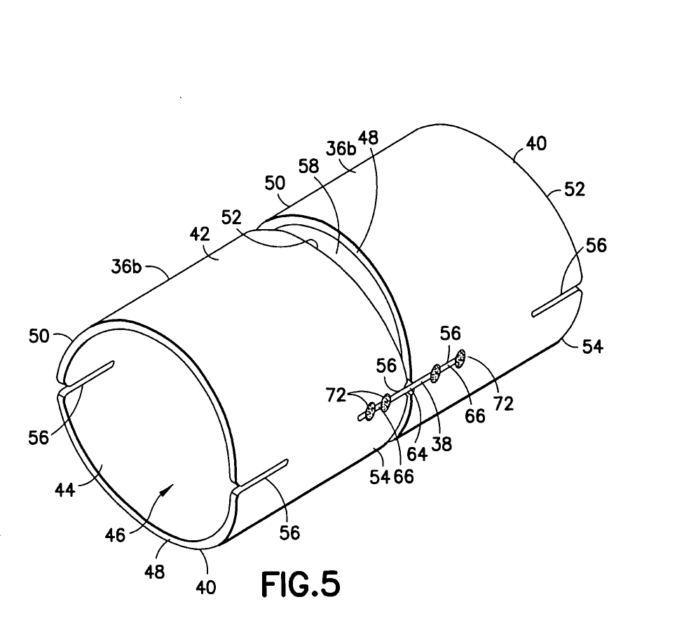

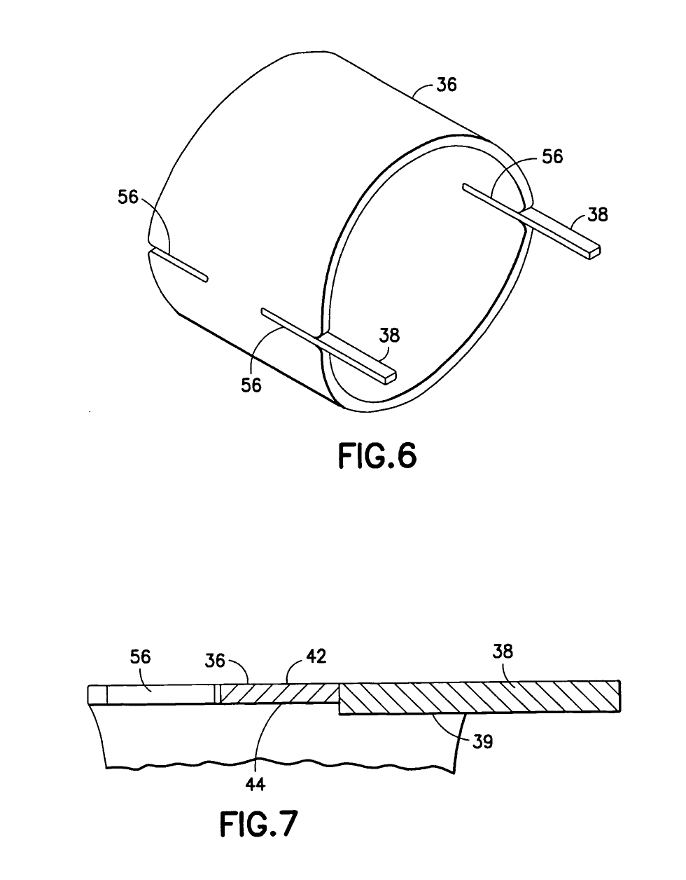

With particular reference to FIGS. 5 and 6, each ring 36 has a wall 40 forming a central channel 46. The wall 40 has an exterior 42 and an interior 44. The wall 40 forms a front side or face 48 at a front end 50 of the ring, a rear side or face 52 at a rear end 54 of the ring. The rings 36b, 36c each comprise connector slots 56 which extend into the front and rear faces 48, 52. In this embodiment, each face 48, 52 has two of the slots 56; one on each opposite side of the face. The proximal and distal rings 36d, g 36a merely comprise their front and rear respective faces having the slots 56.

The slots 56 extend inward into the wall 40 from their respective faces 48, 52. The slots 56 extend entirely through the wall 40 between the exterior and interior 42, 44 of the wall 40. In this embodiment the slots are straight elongate slots. However, in alternate embodiments any suitable shape of the slots could be provided. The front and rear faces 48, 52 taper inward into the wall from the slots 56 to the top and bottom sides of the wall. Thus, tapered clearance gaps 58 (see FIG. 4) are provided between opposing faces 48, 52 of adjacent rings 36. The connectors 38 are flexible to allow the rings 36 to pivot relative to each other at the connectors at the opposing face junctions (at 58) near the slots.

The connectors 38 in this embodiment are leaf springs. Thus, the connectors 38 both connect the rings 36 to each other and apply a spring force between the rings 36 to bias the rings at a home aligned position relative to each other. In the embodiment shown the leaf springs 38 have a straight home position. Thus, the axes of the respective central channels of the rings 36 are co-axially aligned to provide a straight deflection frame at a home position. However, in an alternate embodiment, the leaf springs 38 could have a non-straight shaped home position. Thus, the adjacent rings could be slightly angled relative to each other to provide a curved deflection frame home position.

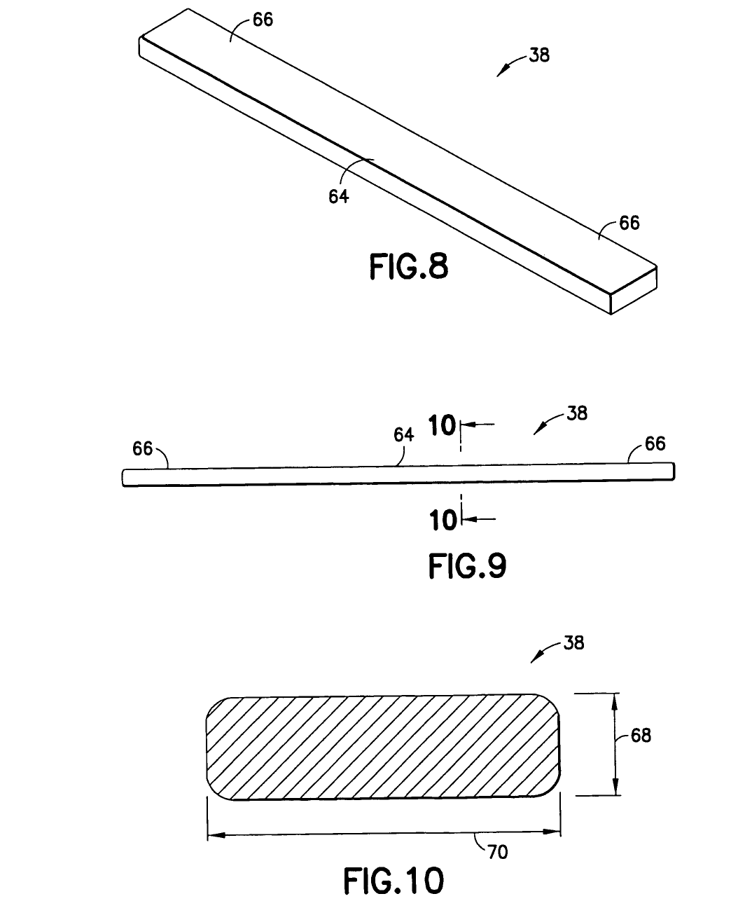

The leaf springs 38 could be made of the same material as the rings 36. The leaf springs 38 have a middle 64 located at the junction of the opposing faces 48, 52 and opposite ends 66 located in the opposing slots 56. As seen in FIGS. 8-10, the leaf spring 38 has a general straight flat wire shape with a rectangular cross section. The height 68 of the leaf spring 38 is about the same height as the slots 56. Thus, the leaf springs 38 can be slid into a pair of aligned slots 56 from the open exterior lateral sides of the slots. The width 70 of the leaf spring 38 is about the same as the width of the wall 40 between the interior 42 and exterior 44. Thus, the interior and exterior sides of the leaf spring do not project significantly beyond the interior 42 and exterior 44. However, in the embodiment shown the width 70 is slightly larger than the width of the slot 56 so the interior facing side 39 of the leaf spring 38 can project slightly inward as seen in FIG. 7.

The ends 66 of the leaf springs 38 are fixedly connected to the rings 36 at the slots 56 by connections. In this embodiment the connections comprise the leaf springs 38 being welded to the rings 36, such as with spot welds 72 shown in FIG. 5 or line weld 73 shown in FIG. 4. However, in alternate embodiments the connections could comprise an additional connection, such as with adhesive, solder, an interlocking physical key shape of the slots and leaf springs, or other fixing connection aid. The welds could be formed by laser welding for example, or ultra sonic welding for plastic material for example.

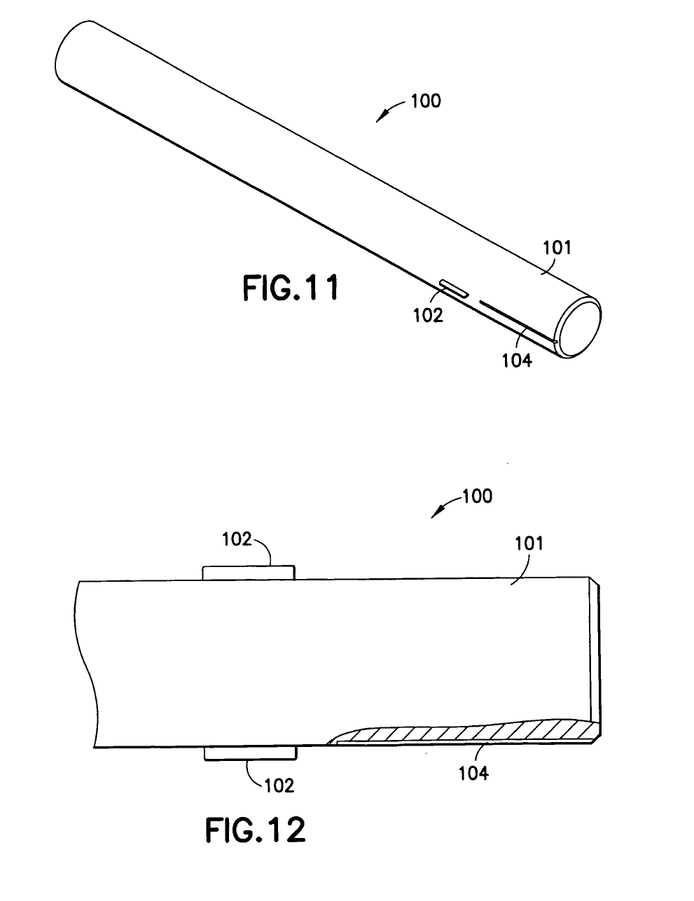

Referring also to FIGS. 11-12, one method of manufacturing the frame 26 can comprise use of a mandrel 100. The mandrel 100 has an end 101 which is sized and shaped to fit inside the channel 46 of one of the rings 36 with the fins 102 being received in the slots 56 at one end of the ring. The mandrel 100 has slot 104 which are aligned with the slots 56 at the other end of the ring. One of the leaf springs 38 can be inserted into the slot 104 and into the slots 56 of adjacent rings on the end 101. The interior facing side 39 (see FIG. 7) of the leaf spring 38 can project into the slot 104. The mandrel 100 can, thus, hold the leaf spring 38 and the rings 36 at a fixed location relative to one another for subsequent welding of the leaf spring to the rings. The mandrel 100 can then be removed. This is only one example of manufacturing the rings and leaf springs together. Other methods could be used.

With the example construction of the frame 26 described above, the connectors 38 do not significantly enlarge the exterior size of the frame, or reduce the available interior space inside the frame 26. The design is also less complicated to manufacture than riveted and ball constructions in conventional endoscope shaft frames. However, because the leaf springs 38 can be made of superelastic alloy, resilient properties can be as good as slotted tube designs, but the frame 26 can have torque resistance superior to a slotted tube endoscope frame.

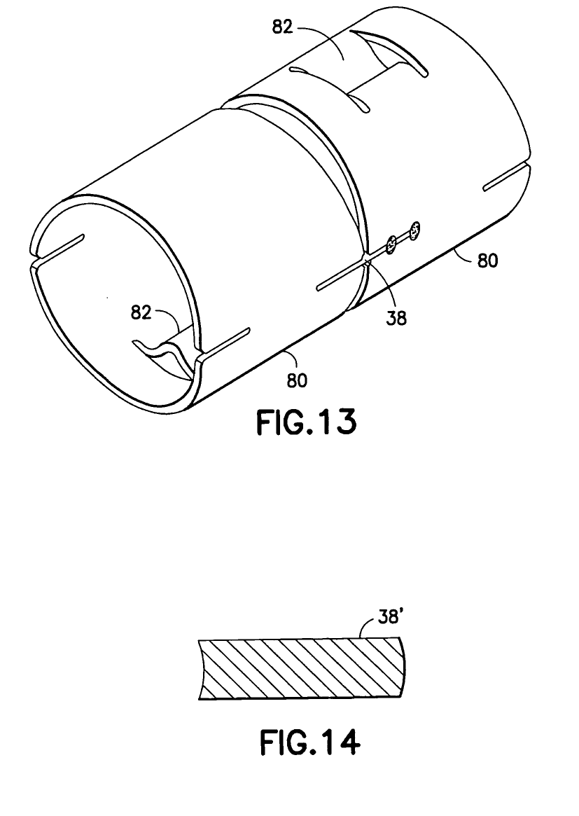

FIG. 13 shows another embodiment wherein the rings 80 comprise indent features 82 on one side of the ring for control cable support. FIG. 14 shows and alternate embodiment with curved interior and exterior sides of the leaf spring.

It should be understood that the foregoing description is only illustrative of the invention. Various alternatives and modifications can be devised by those skilled in the art without departing from the invention. For example, features recited in the various dependent claims could be combined with each other in any suitable combination(s). In addition, features from different embodiments described above could be selectively combined into a new embodiment. Accordingly, the invention is intended to embrace all such alternatives, modifications and variances which fall within the scope of the appended claims.

What is claimed is:

1. An endoscope deflection frame member comprising a one-piece ring having front and rear sides adapted to be connected to adjacent rings to form a deflection section at an end of an endoscope shaft, wherein at least one of the sides comprises slots extending into the at least one side, wherein the slots extend entirely through a wall of the ring between an interior of the wall at a central interior channel of the ring and an opposite exterior of the wall, and wherein the slots are sized and shaped to matingly receive opposite ends of a leaf spring therein to attach the ring to one of the adjacent rings.

2. An endoscope deflection frame member as in claim 1 wherein the slots have an elongate shape extending generally straight into the at least one side.

3. An endoscope deflection frame member as in claim 1 wherein the slots comprise two slots located at opposite lateral sides of the front side, and wherein the front side tapers from the slots to top and bottom sides of the front side.

4. An endoscope deflection frame member as in claim 1 wherein the slots comprise two slots located at opposite lateral sides of the rear side, and wherein the rear side tapers from the slots to top and bottom sides of the rear side.

5. An endoscope deflection frame member as in claim 4 wherein the slots comprise two slots located at opposite lateral sides of the front side, and wherein the front side tapers from the slots to top and bottom sides of the front side.

6. An endoscope deflection section frame comprising:
a deflection frame member as in claim 1;
a second ring connected to the deflection frame member by two of the leaf springs, wherein the second ring forms one of the adjacent rings, wherein the leaf springs each comprise a first end located in a respective one of the slots, and wherein the first ends of the leaf springs are stationarily attached to the deflection frame member at the slots by at least one connection.

7. An endoscope deflection section frame as in claim 6 wherein the connection comprises a weld of the first end of the leaf spring to the deflection frame member.

8. An endoscope frame deflection section as in claim 7 wherein the weld comprises a plurality of spot welds.

9. An endoscope deflection section frame as in claim 6 wherein the leaf springs comprise a general flat elongate shape.

10. An endoscope deflection section frame as in claim 6 wherein the leaf springs comprise a general square or rectangular cross sectional shape.

11. An endoscope deflection section frame comprising:
a plurality of rings, wherein each ring comprises front and rear ends with slots, wherein the slots extend into front and rear sides of the rings entirely through a wall of the ring between an interior of the wall at a central channel of the ring and an opposite exterior of the wall; and
leaf springs connecting the rings to one another, wherein the leaf springs each have opposite ends located in the slots of respective adjacent ones of the rings.

12. An endoscope deflection section frame as in claim 11 wherein a connection of the leaf springs to the rings comprises welds of the leaf springs to the rings at the slots.

13. An endoscope deflection section frame as in claim 12 wherein the welds comprise a plurality of spot welds.

14. An endoscope deflection section frame as in claim 11 wherein the leaf springs comprise a general flat elongate shape.

15. An endoscope deflection section frame as in claim 11 wherein the leaf springs comprise a general square or rectangular cross sectional shape.

16. A method comprising:
positioning a leaf spring between two rings, wherein the rings comprise opposing faces each having a slot, wherein the slots extend entirely through a wall of each respective ring between an interior of the wall at a central channel of the respective ring and an opposite exterior of the wall, wherein the leaf spring comprises opposite ends positioned in the respective slots; and
fixedly attaching the leaf spring to the rings at the slots.

17. A method as in claim 16 wherein fixedly attaching the leaf spring to the rings at the slots comprises welding the leaf spring to the rings.

18. A method as in claim 17 wherein fixedly attaching the leaf spring to the rings at the slots comprises spot welding the leaf spring to the rings at a plurality of locations for each ring.

19. A method as in claim 16 wherein positioning the leaf spring between the two rings comprises sliding the leaf spring into the slots from open exterior lateral sides of the slots.

20. A method as in claim 16 further comprising:
positioning a second leaf spring between the two rings, wherein the opposing faces of the rings each having a second slot, wherein the second slots extend entirely through the wall of each respective ring between the interior of the wall at the central channel of the respective ring and the opposite exterior of the wall, wherein the second leaf spring comprises opposite ends positioned in the respective slots; and
fixedly attaching the second leaf spring to the rings at the second slots, wherein the leaf springs connect the rings to each other an allow the rings to pivot relative to each other at the leaf springs at the opposing faces.
